(19) United States
(12) Patent Application Publication
(10) Pub. No.: US 2023/0240623 A1
(43) Pub. Date: Aug. 3, 2023

(54) MEDICAL INFORMATION DISPLAY APPARATUS, MEDICAL INFORMATION DISPLAY METHOD, AND NON-VOLATILE COMPUTER-READABLE STORAGE MEDIUM STORING THEREIN MEDICAL INFORMATION DISPLAY PROGRAM

(71) Applicant: CANON MEDICAL SYSTEMS CORPORATION, Otawara-shi (JP)

(72) Inventors: Yusuke KANO, Nasushiobara (JP); Anri YAMAZAKI, Nasushiobara (JP); Yohei MURAGUCHI, Nasushiobara (JP)

(73) Assignee: CANON MEDICAL SYSTEMS CORPORATION, Otawara-shi (JP)

(21) Appl. No.: 18/068,671

(22) Filed: Dec. 20, 2022

(30) Foreign Application Priority Data
Feb. 3, 2022 (JP) 2022-015774

(51) Int. Cl. A61B 5/00

(52) U.S. Cl. CPC A61B 5/743; A61B 5/002

(57) ABSTRACT

A medical information display apparatus according to an embodiment includes processing circuitry. The processing circuitry is configured: to obtain time series data related to a predetermined patient; to receive a display time period being a range of data to be displayed from among the time series data; to identify a first state of the patient corresponding to the display time period, on the basis of the time series data; to determine reference data corresponding to a second state different from the first state, on the basis of one selected from between relevant data that is of the same type as the time series data and is relevant to another patient and the time series data; and to cause a display to display time series data corresponding to the display time period and the reference data, so as to be kept in correspondence with each other.

### CROSS-REFERENCE TO RELATED APPLICATIONS

This application is based upon and claims the benefit of priority from Japanese Patent Application No. 2022-015774, filed on Feb. 3, 2022, the entire contents of which are incorporated herein by reference.

### FIELD

Embodiments described herein relate generally to a medical information display apparatus, a medical information display method, and a non-volatile computer-readable storage medium storing therein a medical information display program.

### BACKGROUND

Conventionally, a technique is known by which a trained model generated through machine learning or the like is used for expressing a patient's signs of exacerbation or the like with numerical values, on the basis of time series information related to diagnoses/treatment for the patient. However, simply using an output from the trained model sees a limit to the level of precision for the signs of exacerbation. For this reason, to make a final assessment about the patient's signs of exacerbation, medical providers are required to check the time series information.

For example, there is a conventional technique for displaying, in divided regions, reference information indicating a state of a patient observed at the time of starting home care and time series information generated after the home care is started and accumulated over the course of time. However, to assess the current state of the patient or an exacerbated state of the patient, the reference information is not always helpful in interpreting the time series information. For example, according to the abovementioned conventional technique, because the reference information and the time series information are mutually-different types of diagnosis/treatment information, it is difficult to make comparison between the reference information and the time series information.

Another conventional technique provides a method by which a reference value in general or an abnormal value is displayed. For example, while heart failure is remotely monitored by using a wearable device or the like, it is difficult for medical providers to assess the patient's abnormalities with the conventional technique for the following reasons: Because the monitored patient is already suffering from a disease, the reference value in general (e.g., a reference value for healthy people) may not be useful in correctly evaluating the state of the suffering patient. Further, for instance, there are certain indices (e.g., an activity amount) for which reference values have not clinically been established.

Yet another conventional technique provides a method by which a reference value or an abnormal value is determined for each patient, by using a predetermined algorithm or a trained model. However, even though there is a method for determining the reference value or the abnormal value, the precision level of the determination cannot be 100%.

### BRIEF DESCRIPTION OF THE DRAWINGS

FIG. 1 is a drawing illustrating an exemplary configuration of a medical information display apparatus according to an embodiment, together with a hospital information system and a wearable device;

FIG. 2 is a flowchart illustrating an example of a procedure in a state display process according to the embodiment;

FIG. 3 is a drawing according to the embodiment illustrating a display example of time series data in a display time period when a first state is a caution state and reference data related to a reference time period in a second state;

FIG. 4 is a drawing according to the embodiment illustrating examples of time series data and reference data related to a plurality of pieces of diagnosis/treatment information (heart rates, blood pressure, and the patient's weight);

FIG. 5 is a drawing according to the embodiment illustrating display examples of time series data (text information) in the display time period when the first state is the caution state and reference data (text information) related to the reference time period in the second state;

FIG. 6 is a drawing according to the embodiment illustrating display examples of time series data (image data) in the display time period when the first state is the caution state and reference data (image data) related to the reference time period in the second state;

FIG. 7 is a drawing according to the embodiment illustrating display examples of time series data in the display time period and the reference data related to reference time periods in the second state (a reference state); and

FIG. 8 is a drawing according to the embodiment illustrating examples of choices for the reference time period presented by using a pull-down system (a drop-down system).

### DETAILED DESCRIPTION

A medical information display apparatus according to an embodiment includes processing circuitry. The processing circuitry is configured: to obtain time series data related to a predetermined patient; to receive a display time period being a range of data to be displayed from among the time series data; to identify a first state of the patient corresponding to the display time period, on the basis of the time series data; to determine reference data corresponding to a second state different from the first state, on the basis of one selected from between relevant data that is of the same type as the time series data and is relevant to another patient and the time series data; and to cause a display to display time series data corresponding to the display time period and the reference data, so as to be kept in correspondence with each other.

Exemplary embodiments of a medical information display apparatus, a medical information display method, and a medical information display program will be explained in detail below, with reference to the accompanying drawings. In the following embodiments, some of the elements referred to by using the same reference characters are assumed to perform the same operations, and duplicate explanations thereof will be omitted as appropriate. Further, possible embodiments of the medical information display apparatus, the medical information display method, and the medical information display program of the present disclosure are not limited to the embodiments described below.

### Embodiments

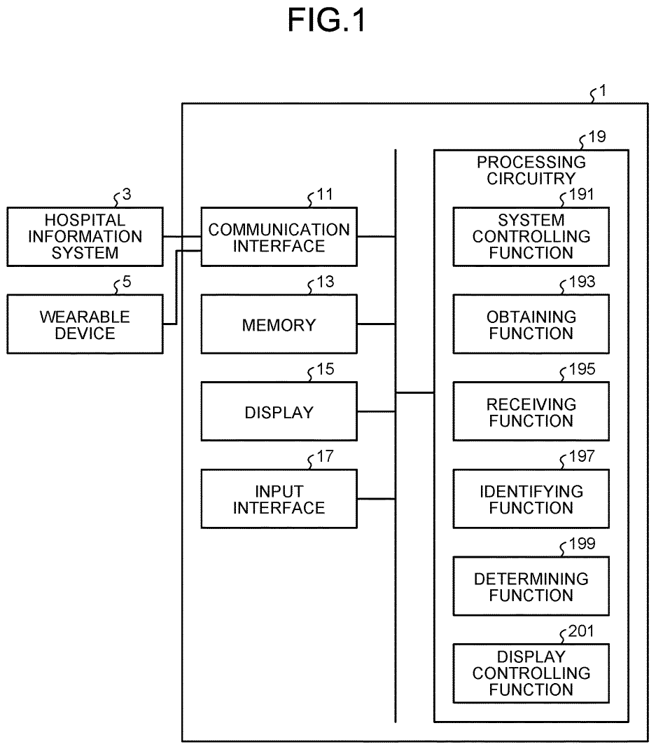

An overall configuration of a medical information display apparatus according to an embodiment will be explained. FIG. 1 is a drawing illustrating an exemplary configuration of a medical information display apparatus 1 according to the embodiment, together with a Hospital Information System (HIS) 3 and a wearable device 5. The medical information display apparatus 1 is connected to the hospital information system 3 and the wearable device 5, via a communication interface 11. At first, the hospital information system 3 and the wearable device 5 connected to the medical information display apparatus 1 will be explained. After that, the medical information display apparatus 1 will be explained. The hospital information system 3 includes an electronic medical record system, a radiology department information system, a medical image diagnosis apparatus, a medical image management system, a clinical examination department information system, and an other department system. The electronic medical record system, the radiology department information system, the medical image diagnosis apparatus, the medical image management system, the clinical examination department information system, and the other department system are connected to one another via a communication network that is wired or wireless.

The electronic medical record system is an information system configured to manage electronic medical records for recording details of diagnoses/treatment. The electronic medical record system includes an electronic medical record server apparatus and an electronic medical record terminal apparatus. The electronic medical record server apparatus is a computer apparatus configured to perform processes related to managing the electronic medical records. The electronic medical record terminal apparatus is used by medical providers (a user) such as a medical doctor, a nurse, and/or the like who input information to and refer to the electronic medical records. The electronic medical record server apparatus and the electronic medical record terminal apparatus are connected to a communication network. According to a user instruction, the electronic medical record terminal apparatus is configured to receive inputs of various types of diagnosis/treatment information, patient information, an examination order, and the like for a patient (which may also be referred to as “examined subject”). In response to the input of the examination order, the electronic medical record server apparatus is configured to transmit the examination order to the radiology department information system, together with patient information of the patient related to the examination order, or the like. When the examination order indicates that a clinical examination be performed on the patient, the electronic medical record server apparatus is configured to transmit the examination order to the clinical examination department information system.

An examination result obtained according to the examination order is brought into association with an electronic medical record related to the patient. In this situation, a clinical doctor who issued an examination request decides a diagnosis result on the basis of the examination result and writes the diagnosis result into the electronic medical record of the patient. The diagnosis result includes information about the name of a diagnosed disease, a stage, severity of the disease stage, and/or the like. For example, according to an instruction from the clinical doctor, the electronic medical record terminal apparatus receives an input of the diagnosis result based on various types of examinations performed for the patient. The diagnosis result is stored into the electronic medical record server apparatus.

The radiology department information system (or a Radiology Information System (RIS)) is an information system configured to manage information in the radiology department of a hospital and includes a radiology department information server apparatus and a radiology department information terminal apparatus. The radiology department information server apparatus is a computer apparatus configured to perform processes related to managing the information in the radiology department. The radiology department information server apparatus and the radiology department information terminal apparatus are connected to a communication network. As an example of operations, the radiology department information server apparatus is configured to receive the examination order from the electronic medical record system and to obtain information about a medical image diagnosis apparatus or the like from the examination order. The radiology department information server apparatus is configured to transmit the examination order to the medical image diagnosis apparatus identified from the examination order. In that situation, when transmitting the examination order to the medical image diagnosis apparatus, the radiology department information server apparatus may add information such as a patient ID and an examination date/time to a Digital Imaging and Communications in Medicine (DICOM) tag or the like. For example, the radiology department information terminal apparatus is used by an image interpreting doctor in the radiology department to create image interpretation reports of medical images.

For example, the medical image diagnosis apparatus is an apparatus configured to acquire medical images, such as an X-ray diagnosis apparatus, an X-ray Computed Tomography (CT) apparatus, or a magnetic resonance imaging apparatus. The medical image diagnosis apparatus is configured to perform an imaging process on the patient, in response to an operation to start the imaging process performed by a technologist or the like, on the basis of the examination order received from the radiology department information system. By performing the imaging process, the medical image diagnosis apparatus is configured to acquire data indicating a state of a tissue in the body of the patient. The medical image diagnosis apparatus is configured to generate a medical image on the basis of the data and to transmit the generated medical image to the medical image management system together with the examination order.

The medical image management system (or a Picture Archiving and Communication System (PACS)) is an information system configured to manage medical images such as X-ray CT images, Magnetic Resonance (MR) images, and/or the like. The medical image management system includes a medical image management server apparatus. The medical image management server apparatus is a computer apparatus configured to perform processes related to managing the medical images and is connected to a communication network. Upon receipt of the medical image and order information from the medical image diagnosis apparatus, the medical image management server apparatus is configured to store therein the medical image and the examination order so as to be kept in association with each other.

The clinical examination department information system (or a Laboratory Information System (LIS)) is an information system configured to manage information in the clinical examination department of the hospital and includes a clinical examination department information server apparatus, a clinical examination department information terminal apparatus, and examination devices. In the clinical examination department information system, a clinical examination technologist performs a specimen examination and/or a physiological function examination, on the basis of the examination order transmitted thereto from the electronic medical record system. Alternatively, the specimen examination may be entrusted to an external examination institution such as a sanitary inspection laboratory. The clinical examination department information server apparatus is a computer apparatus configured to perform processes related to managing information in the clinical examinations. The clinical examination department information terminal apparatus is used by an examination technologist in the clinical examination department or the like. The clinical examination department information server apparatus and the clinical examination department information terminal apparatus are connected to a communication network.

The examination devices are devices related to performing the specimen examination in which a specimen such as blood or urine collected from the patient is analyzed by the clinical examination technologist and the physiological function examination in which a brain wave and/or an electrocardiogram of the patient are acquired. Examples of the specimen examination include a pathology examination, a blood/biochemical examination, a general examination of urine or stool, an immune serum examination, a microbiology examination, and examinations related to blood transfusion and organ transplantation. Examples of the physiological function examination include a brain wave examination, a respiratory function examination, a cardiology examination, and a fundoscopy examination. The two or more examination devices are provided in accordance with different types of specimen examinations and different types of physiological function examinations. At least a part of the specimen examinations may be performed by an external examination institution. On such occasion, the clinical examination department information server apparatus is configured to receive and store therein examination results related to the specimen examinations sent thereto from the external examination institution. Further, the clinical examination department information server apparatus is configured to transmit examination results to the electronic medical record system.

The other department system may be, for example, a surgery department system, a rehabilitation department system, or a dialysis department system. Similarly to the radiology department information system and the clinical examination department information system, in the other department system also, a surgery implementation report, a rehabilitation implementation report, or the like is generated in accordance with an order issued by a medical doctor while using the electronic medical record terminal apparatus. The other department system is configured to transmit the generated surgery implementation report or rehabilitation implementation report to the electronic medical record system.

The wearable device 5 is attached to the patient, for example. The wearable device 5 is configured to sequentially acquire biological information related to the patient. The wearable device 5 is realized by using an existing activity meter, an existing vital sign meter, or the like. In this situation, the acquired biological information corresponds to time series data and represents any of the following, for example: activity amounts of the patient such the numbers of walking steps, consumed calories, a sleep state, and oxygen saturation levels; and vital signs such as heart rates, respiratory rates, body temperatures, and blood pressure levels. Possible examples of the biological information acquired by the wearable device 5 are not limited to the activity amounts and the vital signs and may include the patient's weight, for example. The wearable device 5 is configured to transmit the sequentially-acquired biological information to the medical information display apparatus 1 via a communication network.

The medical information display apparatus 1 is connected to the hospital information system 3 and the wearable device 5 via the communication interface 11. Alternatively, the medical information display apparatus 1 may be incorporated in the hospital information system 3. In another example, the medical information display apparatus 1 may be realized as a client-server system. The medical information display apparatus 1 illustrated in FIG. 1 includes the communication interface 11 (a communication unit), a memory 13 (a storage unit), a display 15 (a display unit), an input interface 17 (an input unit), and processing circuitry 19 (a processing unit). Data communication among the communication interface 11, the memory 13, the display 15, the input interface 17, and the processing circuitry 19 is performed via a bus.

The communication interface 11 is configured to perform data communication with the hospital information system 3. Although the communication standard used between the communication interface 11 and the hospital information system 3 may be any standard, it is acceptable to use, for example, one or both of Health Level 7 (HL7) and DICOM. Further, the communication interface 11 is configured to receive the biological information acquired by the wearable device 5 from the wearable device 5.

The memory 13 is a storage apparatus such as a Hard Disk Drive (HDD), a Solid State Drive (SSD), or an integrated circuitry storage apparatus configured to store therein various types of information. For example, the memory 13 is configured to store therein various types of data related to implementation of an obtaining function 193, a receiving function 195, an identifying function 197, a determining function 199, and a display controlling function 201. The various types of data and the functions will be explained later. By employing the obtaining function 193, the memory 13 is configured to store therein the time series data related to the predetermined patient and acquired from one or both of the hospital information system 3 and the wearable device 5. The time series data includes any of the following kept in association with the electronic medical record of the patient: clinical information such as various types of examination results, medical images, and image interpretation reports; and biological information. In other words, the time series data corresponds to data indicating changes in diagnosis/treatment information of the patient over the course of time. Other than being an HDD or an SSD, the memory 13 may be a drive apparatus configured to read and write various types of information from and to a portable storage medium such as a Compact Disc (CD), a Digital Versatile Disc (DVD), or a flash memory, or a semiconductor memory element such as a Random Access Memory (RAM). Further, the memory 13 is configured to store therein a control program related to the present embodiment.

The display 15 is configured to display various types of information. For example, the display 15 is configured to output a Graphical User Interface (GUI) or the like used for receiving various types of operations from the user. For example, as the display 15, it is possible to use, as appropriate, a Liquid Crystal Display (LCD) device, a Cathode Ray Tube (CRT) display, an Organic Electroluminescence Display (GELD) device, a plasma display, or other arbitrary displays. Further, the display 15 may be of a desktop type or may be configured by using a tablet terminal or the like capable of wirelessly communicating with the main body of the medical information display apparatus 1. The specifics of what is displayed by the display 15 will be explained later.

The input interface 17 is configured to receive various types of input operations from the user, to convert the received input operations into electrical signals, and to output the electrical signals to the processing circuitry 19. For example, the input interface 17 is configured to receive selecting instructions and various types of conditions from the user. As the input interface 17, for example, it is possible to use any of the following as appropriate: a mouse, a keyboard, a trackball, a switch, a button, a joystick, a touchpad, and a touch panel display. In the present embodiment, the input interface 17 does not necessarily have to include physical operation component parts such as the mouse, the keyboard, the trackball, the switch, the button, the joystick, the touchpad, the touch panel display, and/or the like. For instance, possible examples of the input interface 17 include electrical signal processing circuitry configured to receive an electrical signal corresponding to an input operation from an external input device provided separately from the apparatus and to output the electrical signal to the processing circuitry 19. Alternatively, the input interface 17 may be configured by using a tablet terminal or the like capable of wirelessly communicating with the main body of the medical information display apparatus 1.

The processing circuitry 19 is configured to control operations of the entirety of the medical information display apparatus 1 in accordance with the electrical signals of the input operations output from the input interface 17. For example, as hardware resources thereof, the processing circuitry 19 includes a processor such as a Central Processing Unit (CPU), a Micro Processing Unit (MPU), and/or a Graphics Processing Unit (GPU), as well as a memory such as a Read Only Memory (ROM), a RAM, and/or the like. Further, the processing circuitry 19 may be realized by using an Application Specific Integrated Circuit (ASIC), a Field Programmable Gate Array (FPGA), one of other types of Complex Programmable Logic Devices (CPLDs), a Simple Programmable Logic Device (SPLD), or the like.

The processing circuitry 19 includes a system controlling function 191, the obtaining function 193, the receiving function 195, the identifying function 197, the determining function 199, and the display controlling function 201. As for the system controlling function 191, the obtaining function 193, the receiving function 195, the identifying function 197, the determining function 199, and the display controlling function 201, the functions thereof are stored in the memory 13 in the form of computer-executable programs. By using one or more processors configured to execute programs loaded into a memory thereof, the processing circuitry 19 is configured to implement the system controlling function 191, the obtaining function 193, the receiving function 195, the identifying function 197, the determining function 199, and the display controlling function 201.

In other words, the processing circuitry 19 corresponds to a processor configured to realize the functions corresponding to the programs by reading and executing the programs from the memory 13. That is to say, the processing circuitry 19 that has read the programs has the functions corresponding to the read programs. In this situation, the functions (191, 193, 195, 197, 199, and 201) do not necessarily have to be realized by the single piece of processing circuitry (the processing circuitry 19). It is also acceptable to structure the processing circuitry 19 by combining together a plurality of independent processors, so that the functions (191, 193, 195, 197, 199, and 201) are realized as a result of the processors executing the programs. The system controlling function 191, the obtaining function 193, the receiving function 195, the identifying function 197, the determining function 199, and the display controlling function 201 are examples of a system controlling unit, an obtaining unit, a receiving unit, an identifying unit, a determining unit, and a display controlling unit, respectively.

By employing the system controlling function 191, the processing circuitry 19 is configured to control the functions of the processing circuitry 19, on the basis of the input operations received from the user via the input interface 17. More specifically, the processing circuitry 19 is configured to read the control program stored in the memory 13, to load the read control program into a memory within the processing circuitry 19, and to control various functional units in the medical information display apparatus 1 according to the loaded control program. The processing circuitry 19 is configured to control the display 15 regarding various types of displays realized by the display 15. The obtaining function 193, the receiving function 195, the identifying function 197, the determining function 199, and the display controlling function 201 realized by the processing circuitry 19 will be explained later, while following a procedure in a process (hereinafter, “state display process”) of causing the display 15 to display reference data and the time series data of the patient in such a manner that medical providers are able to easily understand a state of the patient.

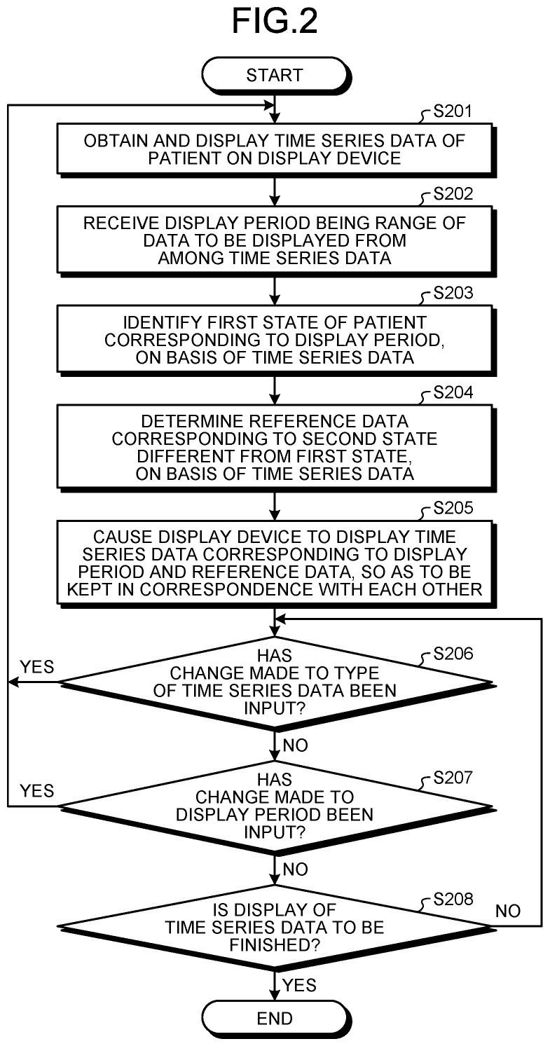

Overall configurations of the medical information display apparatus 1 and the hospital information system 3 have thus been explained. Next, a procedure in the state display process will be explained. FIG. 2 is a flowchart illustrating an example of the procedure in the state display process.

### State Display Process

### Step S201:

By employing the obtaining function 193, the processing circuitry 19 obtains time series data related to the predetermined patient. More specifically, the obtaining function 193 obtains the biological information of the patient transmitted from the wearable device 5 via the communication interface 11. Further, according to an instruction of a medical provider received via the input interface 17, the obtaining function 193 obtains time series data related to the predetermined patient from the hospital information system 3.

By employing the display controlling function 201, the processing circuitry 19 causes the display 15 to display the time series data. For example, the display controlling function 201 causes the display 15 to display a part of the time series data in a display time period (hereinafter, “display period”) corresponding to the type of the time series data. More specifically, when having obtained time series data of heart rates acquired four times a day, the display controlling function 201 causes the display 15 to display the time series data of the heart rates over the past ten days preceding the most recent heart rate acquisition time. In that situation, when a type of time series data (e.g., one of various types of items in the biological information or various types of medical data in a time series related to the patient) is selected according to an instruction of the medical provider received via the input interface 17, the display controlling function 201 causes the display 15 to display the time series data corresponding to the selected type and acquired in a display period corresponding to the type.

Further, when the display period of the time series data is changed according to an instruction of the medical provider received via the input interface 17, the display controlling function 201 causes the display 15 to display the time series data corresponding to the post-change display period.

### Step S202:

By employing the receiving function 195, the processing circuitry 19 receives a display period, which is a range of data to be displayed from among the time series data. More specifically, when the time series data is displayed on the display 15 as a result of step S201, the receiving function 195 receives the display period of the time series data currently displayed on the display 15, either from the display 15 or the display controlling function 201.

### Step S203:

By employing the identifying function 197, the processing circuitry 19 identifies a first state of the patient corresponding to the display period, on the basis of the time series data. More specifically, the identifying function 197 identifies the first state, by using at least one piece of diagnosis/treatment information in the time series data. For example, the diagnosis/treatment information includes one or both of: a subjective symptom of the patient related to the time series data; and details of a diagnosis/treatment event related to the patient.

For example, when the patient has a heart failure symptom, the subjective symptom may be whether edema is present or absent. In that situation, the identifying function 197 is configured to determine that the time period in which edema is “present” and the time period in which edema is “absent” correspond to mutually-different patient states. More specifically, when the patient's subjective symptom in the display period indicates that edema is “present”, the identifying function 197 identifies the first state as an exacerbated state. On the contrary, when the patient's subjective symptom in the display period indicates that edema is “absent”, the identifying function 197 identifies the first state as a stable state.

For example, the diagnosis/treatment event is an event of the patient being admitted into and/or discharged from the hospital related to the displayed time series data. For example, in the display period, the identifying function 197 identifies the first state by defining a time period before and after the event of the patient being discharged from the hospital as a “stable state”, the time period immediately after the event of the patient being admitted into the hospital as an “exacerbated state”, and the rest of the time period as a “caution state”.

In this situation, the process of identifying the first state performed by the identifying function 197 is not limited to the method described above. For example, the identifying function 197 may identify the first state, by inputting a plurality of pieces of diagnosis/treatment information to a trained model configured to receive an input of a plurality of pieces of diagnosis/treatment information in the display period and to output the first state. The trained model is stored in advance in the memory 13. For example, a trained model configured to predict an exacerbated state of heart failure may be configured to output the “exacerbated state”, the “stable state”, and the “caution state”. In another example, the trained model may be configured to output a predicted probability related to a state of the patient. In that situation, the identifying function 197 may convert the predicted probability output from the trained model into a patient's state corresponding to one of multiple levels (e.g., three levels such as the “exacerbated state”, the “stable state”, and the “caution state”; or five levels such as an “exacerbated state”, a “semi-exacerbated state”, a “caution state”, a “semi-stable state”, and a “stable state”) in accordance with the magnitude of the value of the predicted probability.

### Step S204:

By employing the determining function 199, the processing circuitry 19 determines reference data corresponding to a second state different from the first state, on the basis of the time series data. More specifically, the determining function 199 determines the reference data, by using at least one piece of diagnosis/treatment information in the time series data. From among the time series data different from that in the display period, the reference data includes time series data corresponding to the second state. In the time series data, the time period corresponding to the reference data will hereinafter be referred to as “reference time period” (or simply “reference period”).

For example, in the display period of the time series data currently displayed on the display 15, when the first state is the “exacerbated state”, the determining function 199 determines the second state as the “stable state” and determines the time series data corresponding to the “stable state” as the reference data. In this situation, among the time series data, the time period included in the “stable state” serves as the reference period. In another example, in the display period of the time series data currently displayed on the display 15, when the first state is the “stable state”, the determining function 199 determines the second state as the “exacerbated state” and determines the time series data corresponding to the “exacerbated state” as the reference data. In this situation, among the time series data, the time period included in the “exacerbated state” serves as the reference period.

More specifically, when the patient has a heart failure symptom, the subjective symptom may be whether edema is present or absent. In that situation, when the patient's subjective symptom in the display period indicates that edema is “present”, the determining function 199 determines the time series data included in the time period in which edema is “absent”, as the reference data. In that situation, the time period in which edema is “absent” corresponds to the reference period. On the contrary, when the patient's subjective symptom in the display period indicates that edema is “absent”, the determining function 199 determines the time series data included in the time period in which edema is “present”, as the reference data. In that situation, the time period in which edema is “present” corresponds to the reference period.

Further, for example, when the display period is a time period (the stable state) before and after the event of the patient being discharged from the hospital, the determining function 199 determines the time series data corresponding to the time period (the exacerbated state) immediately after the event of the patient being admitted into the hospital and to the time period (hereinafter, “non admittance/discharge period”) different from the events of the patient being admitted into and discharged from the hospital, as the reference data. In that situation, the reference period is the time periods corresponding to the time period (the exacerbated state) immediately after the event of the patient being admitted into the hospital and to the non admittance/discharge period which corresponds to the caution state.

As another example, when the display period is a time period (the exacerbated state) before and after the event of the patient being admitted into the hospital, the determining function 199 determines the time series data corresponding to the time period (the stable state) immediately after the event of the patient being discharged from the hospital and to the non admittance/discharge period (the caution state), as the reference data. In that situation, the reference period is the time periods corresponding to the time period (the stable state) immediately after the event of the patient being discharged from the hospital and to the non admittance/discharge period (the caution state).

As yet another example, when the display period is the non admittance/discharge period (the caution state), the determining function 199 determines the time series data corresponding to the time period (the exacerbated state) immediately after the event of the patient being admitted into the hospital and to the time period (the stable state) immediately after the event of the patient being discharged from the hospital, as the reference data. In that situation, the reference period is the time periods corresponding to the time period (the exacerbated state) immediately after the event of the patient being admitted into the hospital and to the time period (the stable state) immediately after the event of the patient being discharged from the hospital.

However, the process of determining the second state is not limited to the method described above. For example, the determining function 199 may determine the second state, by inputting a plurality of pieces of diagnosis/treatment information to the abovementioned trained model configured to receive an input of a plurality of pieces of diagnosis/treatment information in a time period different from the display period and to output the second state. In that situation, the determining function 199 determines the time series data corresponding to the second state, as the reference data.

Further, the determining function 199 may determine the reference data, while regarding a state farthest away from the first state in a diagnosis/treatment sense as the second state. For example, when the medical provider is able to think of a plurality of states among which he/she can determine superior/inferior ranking, the determining function 199 may be configured to determine the time period corresponding to the patient's state farthest away, in view of the state, from the patient's state in the display period, as the reference period. For example, a state farthest away from the “stable state” is determined as the “exacerbated state”. In other words, when the “stable state” is identified as the first state, the determining function 199 determines the time series data related to the “exacerbated state”, which is the state farthest away from the “stable state”, as the reference data.

More specifically, when there are two or more intermittent reference periods that can be considered or when a reference period lasts for a long period of time, the determining function 199 may determine the reference period on the basis of a predetermined standard. The predetermined standard may be defined so that, for example, if the second state lasts for one day or longer, the one-day period closest to the current display period is set as the reference period. In that situation, the determining function 199 is configured to determine the time series data included in the reference period as the reference data. In these situations, the time width of the reference period may be fixed or may dynamically be changed in accordance with the acquisition intervals of the plurality of pieces of data in the time series data displayed on the display 15 or the types of the diagnosis/treatment information displayed on the display 15.

In another example, the determining function 199 may be configured to determine a plurality of second states and to determine the reference data, on the basis of differences between the first state and the plurality of second states and temporal closeness between a plurality of time periods respectively corresponding to the plurality of second states and the display period. In other words, when there are two or more time periods related to the second state in the time series data, the determining function 199 may determine the reference period while taking into consideration both temporary closeness/remoteness with the display period and state-specific closeness/remoteness with the first state, so as to determine the time series data included in the reference period as the reference data. In that situation, the determining function 199 is configured to determine the reference period, on the basis of a value of an index based on a weight applied in accordance with the temporary closeness/remoteness with the display period and a weight applied in accordance with the state-specific closeness/remoteness with the first state. As for the weights being applied, for example, larger weights are applied for being temporally closer to the display period and for being more remote, in terms of the state, from the first state.

### Step S205:

By employing the display controlling function 201, the processing circuitry 19 causes the display 15 to display the time series data corresponding to the display period and the reference data, so as to be kept in correspondence with each other. In this situation, the time series data in the display period and the reference data are mutually the same type of diagnosis/treatment information. Pieces of diagnosis/treatment information of mutually the same type do not necessarily have to be a completely match and may be pieces of information having similar meanings to each other. For example, blood pressure measured by the patient himself/herself at home or the like while using a home-use automatic blood pressure monitor is different, strictly speaking, from invasive blood pressure taken from an arterial line during hospitalization. However, both types of blood pressure may equally be handled as blood pressure. The diagnosis/treatment information of the same type may be determined, distinguished, and identified by the identifying function 197, for example, on the basis of the purpose of the examination, the examined items, the examined site, additional information related to the examination, and/or the like indicated in the diagnosis/treatment information.

Further, the diagnosis/treatment information displayed as the time series data and the reference data may be two or more types of information. Further, the diagnosis/treatment information does not necessarily have to be numerical value data such as activity amounts and vital signs, and may be text information (e.g., writing in the medical records, nurse records), image information (medical images, as well as Portable Document Format (PDF) documents, etc.), or the like.

Further, the diagnosis/treatment information displayed as the time series data and the reference data is not limited to actual data obtained from measuring and/or examination processes and may be, for example, diagnosis/treatment information estimated by a trained model or the like. For example, although Brain Natriuretic Peptide (BNP), which is a typical biomarker for heart failure, is not usually measured during home care, the display controlling function 201 may display a BNP value during home care estimated from other diagnosis/treatment information. For example, at step S201, the obtaining function 193 may read a trained model stored in the memory 13 from the memory 13, so as to obtain the BNP value from the trained model, by inputting an oxygen saturation level and a heart rate acquired by the wearable device 5 to the trained model read from the memory 13.

In an example, the display controlling function 201 may be configured to cause the display 15 to further display the second state. Also, when there are a plurality of second states in the time series data, the display controlling function 201 may cause the display 15 to display the data in a time period closest to the display period among the reference data.

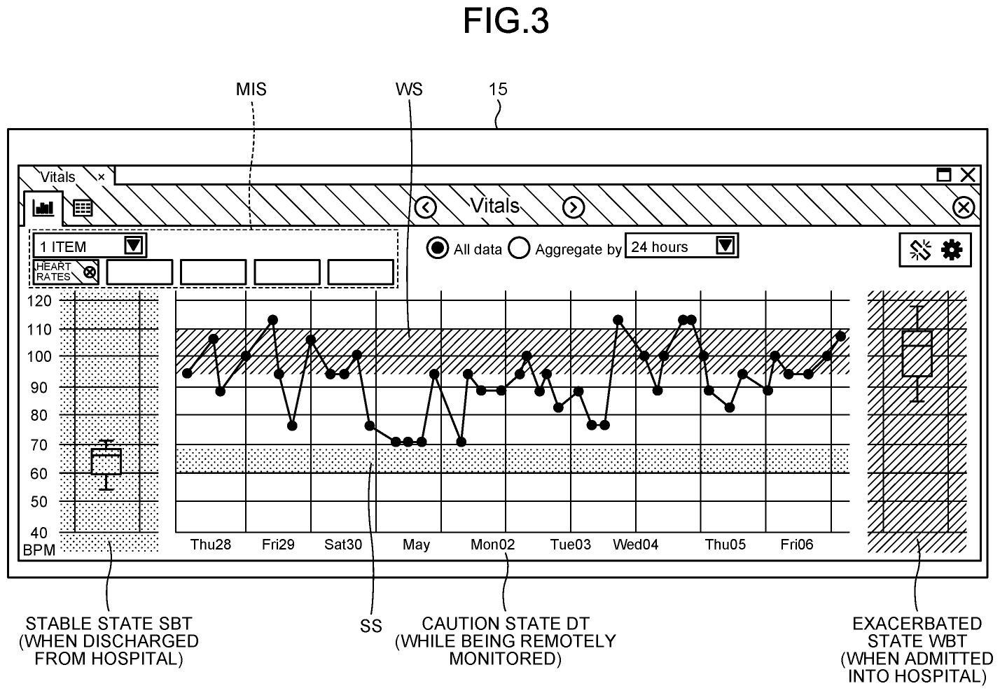

FIG. 3 is a drawing illustrating a display example of time series data in a display period DT (while being remotely monitored) when the first state is the caution state and the reference data related to the reference period while being in the second state (the stable state (at the time of being discharged from the hospital) and the exacerbated state (at the time of being admitted into the hospital)). As illustrated in FIG. 3, the horizontal axes of a reference period SBT for the stable state, a reference period WBT for the exacerbated state, and the display period DT express time. In this situation, the horizontal axes of the reference period SBT for the stable state, the reference period WBT for the exacerbated state, and the display period DT may be scaled as appropriate, in accordance with the acquisition intervals of the time series data. Further, the vertical axes of the reference period SBT for the stable state, the reference period WBT for the exacerbated state, and the display period DT express axes related to values of the diagnosis/treatment information in the time series data. In this situation, the vertical axes of the reference period SBT for the stable state, the reference period WBT for the exacerbated state, and the display period DT may be scaled as appropriate, in accordance with the values of the time series data in the display period DT.

As illustrated in FIG. 3, the display 15 is configured to display the heart rates, as diagnosis/treatment information from the time series data in the display period DT. Further, as illustrated in FIG. 3, the reference data corresponding to the stable state (at the time of being discharged from the hospital) and the exacerbated state (at the time of being admitted into the hospital) is plotted in box-and-whisker charts of the heart rates, in the reference period SBT for the stable state and the reference period WBT for the exacerbated state, on either end of the display period DT. Possible display modes of the diagnosis/treatment information in the reference period SBT for the stable state and the reference period WBT for the exacerbated state are not limited to box-and-whisker charts. It is also acceptable to display the time series data in the reference period SBT for the stable state and in the reference period WBT for the exacerbated state.

Further, as illustrated in FIG. 3, over the display period DT, the second state (a stable state SS and an exacerbated state WS in FIG. 3) is displayed, while being superimposed in a display mode corresponding to the second state. In other words, by employing the display controlling function 201, the processing circuitry 19 is configured to cause the display 15 to display a relative positional relationship of the second state with the time series data in the display period DT so as to be superimposed on the display period DT, by using the display mode corresponding to the second state. (Although the mutually-different hatching patterns are used in FIG. 3, possible embodiments are not limited to this example, and hues or the like may be used.)

Further, as illustrated in FIG. 3, as for the display mode of the background of the second state (the stable state SS and the exacerbated state WS) in the reference period, the display is of the same type as the display mode (e.g., a hue) used for the second state (the stable state SS and the exacerbated state WS) superimposed on the display period DT. With these arrangements, the medical provider is able to easily recognize the relative positional relationship among the reference data in the display period DT. Further, the display layout presented in FIG. 3 is merely an example and may arbitrarily be changed.

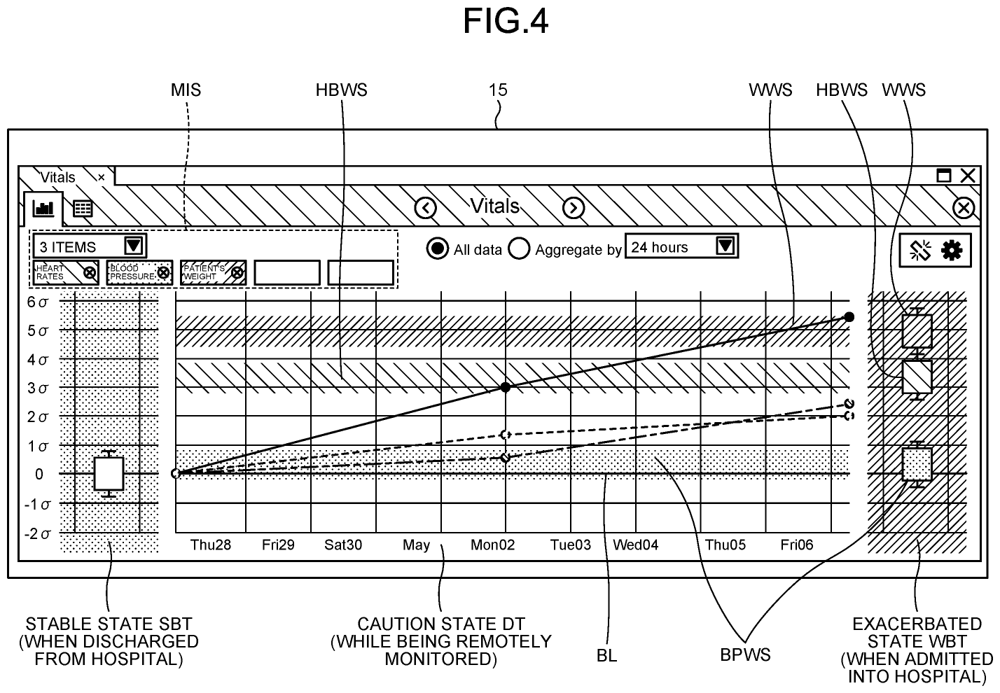

FIG. 4 is a drawing illustrating examples of time series data and reference data related to a plurality of pieces of diagnosis/treatment information (heart rates, blood pressure, and the patient's weight). In the display example of FIG. 4, because the plurality of mutually-different pieces of diagnosis/treatment information are displayed, the time series data of the plurality of pieces of diagnosis/treatment information (the heart rates, the blood pressure, and the patient's weight) are each standardized. The standardization of the time series data is realized by the obtaining function 193, for example, after the time series data is acquired. Alternatively, the standardization of the time series data may be performed by any of the other functions illustrated in FIG. 1 or may be performed by a standardizing function (a standardizing unit) separately and newly provided in the processing circuitry 19.

The standardization of the time series data may be carried out, for example, by standardizing a mean value (or an average value) included in a reference period BT for the stable state as 0 and standardizing a standard deviation as 1, with respect to each of the plurality of pieces of diagnosis/treatment information. The mean value standardized in the plurality of pieces of diagnosis/treatment information is indicated as a reference line BL in the display period DT illustrated in FIG. 4. In that situation, the vertical axes of the reference period SBT for the stable state, the reference period WBT for the exacerbated state, and the display period DT are displayed with standard deviations, for example.

In FIG. 4, a range WWS of the patient's weight in the exacerbated state is superimposed on the display period DT and is plotted in a box-and-whisker chart in the reference period WBT for the exacerbated state. Further, fluctuation of the standardized patient's weight in the display period DT is indicated by the solid line. In FIG. 4, a range HBWS of the heart rates in the exacerbated state is superimposed on the display period DT and is plotted in a box-and-whisker chart in the reference period WBT for the exacerbated state. In addition, fluctuation of the standardized patient's weight in the display period DT is indicated by the broken line. In FIG. 4, a range BPWS of the blood pressure levels in the exacerbated state is superimposed on the display period DT and is plotted in a box-and-whisker chart in the reference period WBT for the exacerbated state. In addition, in FIG. 4, the range BPWS of the blood pressure in the exacerbated state is superimposed on the display period DT and is plotted in a box-and-whisker chart in the reference period WBT for the exacerbated state. Further, fluctuation of the standardized blood pressure in the display period DT is indicated by the dotted line.

From the display example presented in FIG. 4, the medical provider is able to easily recognize that, among the three pieces of diagnosis/treatment information (the heart rates, the blood pressure, and the patient's weight), the patient's weight changed so as to reach an exacerbated state during the display period DT. In other words, in the display example presented in FIG. 4, it is implied that the patient's weight is most important as an index for determining the exacerbated state, while the heart rates are second most important, whereas the blood pressure is not so important. In the display period DT illustrated in FIG. 4, among the time series data, the chronological fluctuation in the patient's weight reached the range WWS corresponding to the exacerbated state. For this reason, the medical provider is able to understand that the state of the patient was gradually exacerbated in the display period DT. The display layout presented in FIG. 4 is merely an example and may arbitrarily be changed.

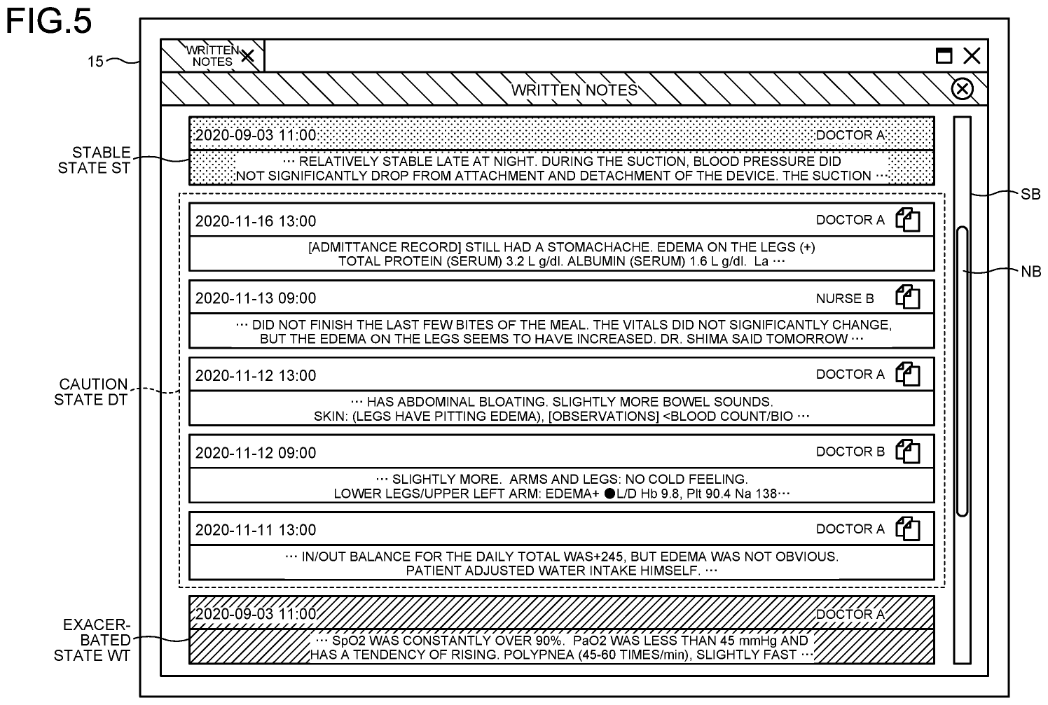

FIG. 5 is a drawing illustrating display examples of time series data (text information) in the display period DT when the first state is the caution state and reference data (text information) related to the reference period in the second state (the stable state and the exacerbated state). As illustrated in FIG. 5, the text information related to the stable state ST and the text information related to the exacerbated state WT are displayed on either end of the display period DT, for example. In the display example in FIG. 5, the text information related to the stable state ST is displayed at the top end of the display period DT. Further, in the display example in FIG. 5, the text information related to the exacerbated state WT is displayed at the bottom end of the display period DT. However, the display layout of the text information related to the stable state ST, the text information related to the exacerbated state WT, and the time series data in the display period DT is merely an example and may arbitrarily be changed.

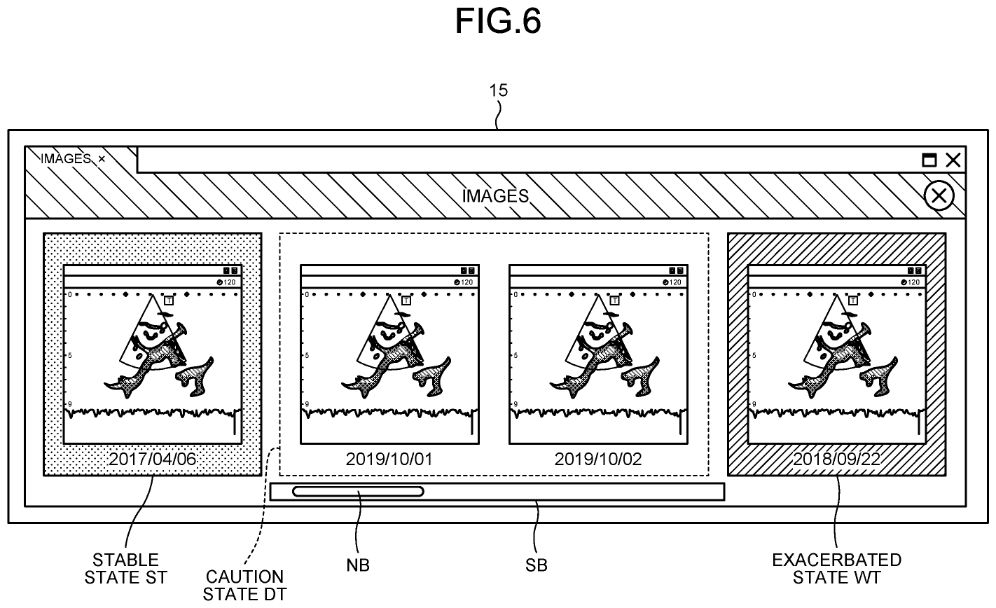

FIG. 6 is a drawing illustrating display examples of time series data (image data) in the display period DT when the first state is the caution state and reference data (image data) related to the reference period in the second state (the stable state and the exacerbated state). As illustrated in FIG. 6, the image data related to the stable state ST and the image data related to the exacerbated state WT are displayed on either end of the display period DT, for example. In the display example in FIG. 6, the image data related to the stable state ST is displayed at the left end of the display period DT. Further, in the display example in FIG. 6, the image data related to the exacerbated state WT is displayed at the right end of the display period DT. However, the display layout of the image data related to the stable state ST, the image data related to the exacerbated state WT, and the time series data in the display period DT is merely an example and may arbitrarily be changed.

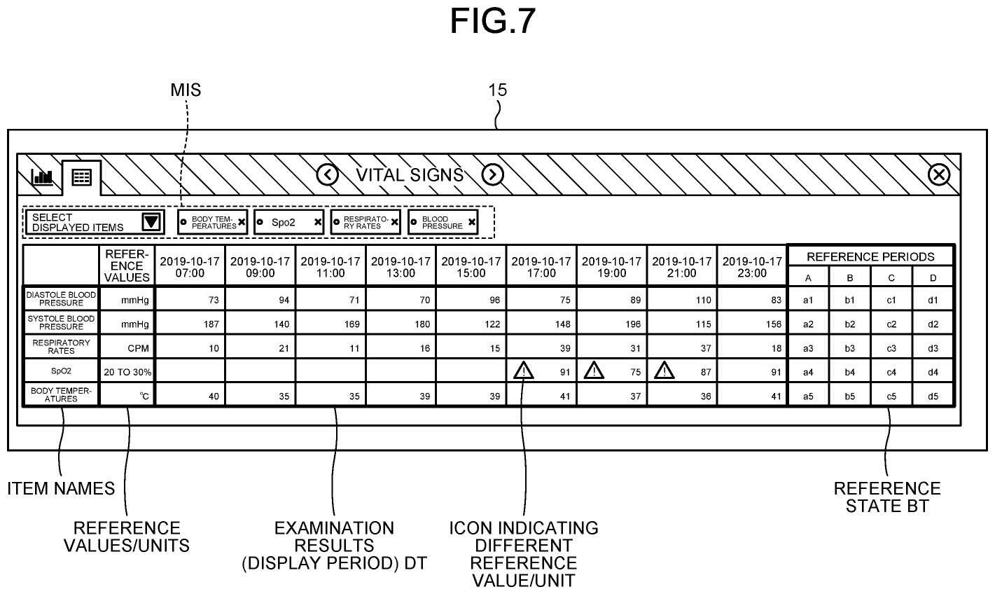

FIG. 7 is a drawing illustrating display examples of time series data in the display period DT and reference data (a1 to a5, b1 to b5, c1 to c5, and d1 to d5) respectively related to a plurality of reference periods (A, B, C, and D) in the second state (the reference state) displayed in a graph format. As illustrated in FIG. 7, the display controlling function 201 is configured to cause the display 15 to display the time series data in the reference state BT so as to be positioned adjacent to the time series data in the display period DT. Although FIG. 7 illustrates the example of displaying the time series data in the reference period, possible embodiments are not limited to this example. For instance, it is also acceptable, on the basis of the time series data over the reference period, to display a first quantile value, a second quantile value (a mean value), a third quantile value, a maximum value, a minimum value, and/or the like with respect to each of the pieces of diagnosis/treatment information. Further, in a table format, for example, each of the various types of items in the diagnosis/treatment information and a reference value with a unit thereof may be displayed so as to be positioned adjacent to the display period DT. Further, as illustrated in FIG. 7, when the time series data in the display period DT has mutually-different reference values and units thereof, icons indicating this situation may be displayed.

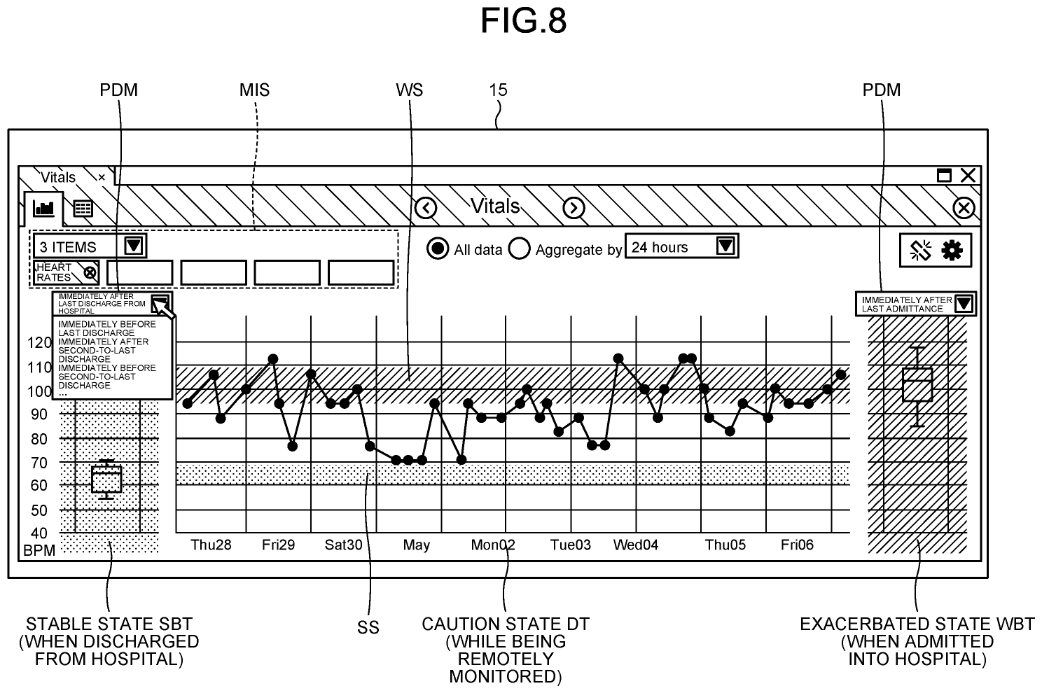

Further, when a plurality of reference periods have been determined with regard to the second state, one of the reference periods may be selected according to an instruction of the medical provider received via the input interface 17. FIG. 8 is a drawing illustrating examples of choices for the reference period presented by using a pull-down system (a drop-down system). As illustrated in FIG. 8, when a pull-down menu (a drop-down menu) PDM related to the reference period is selected via a cursor, the display controlling function 201 is configured to display the plurality of reference periods in the order based on a predetermined standard. In FIG. 8, the plurality of reference periods chronologically earlier than the display period DT are displayed in ascending order of closeness to the display period DT. The display controlling function 201 is configured to display, together with the display period DT, the reference data corresponding to one of the reference periods selected according to an instruction of the medical provider received via the input interface 17.

As another example, the types of the diagnosis/treatment information to be displayed may automatically be selected. For instance, when the “stable state” and the “exacerbated state” are displayed as the reference periods together with the time series data, the determining function 199 may be configured to automatically detect the type of time series data (the diagnosis/treatment information) exhibiting the largest difference in tendencies of the time series data related to the display period DT, together with the reference period. In that situation, the display controlling function 201 is configured to cause the display 15 to display the type of time series data corresponding to the determined diagnosis/treatment information, together with the display period DT and the reference period.

### Step S206:

When a change made to the type of diagnosis/treatment information displayed in the display period DT is input according to an instruction of the medical provider received via the input interface 17 (step S206: Yes), the time series data of the post-change diagnosis/treatment information is obtained by the obtaining function 193, so that the processes at step S201 and thereafter are repeatedly performed. For example, the instruction to change the type of diagnosis/treatment information may suggest adding, deleting, or switching the diagnosis/treatment information by using the selection tabs MIS for the diagnosis/treatment information illustrated in FIGS. 3, 4, and 7. When no change made to the type of diagnosis/treatment information displayed in the display period DT is input according to an instruction of the medical provider received via the input interface 17 (step S206: No), the process at step S207 will be performed.

### Step S207:

When a change made to the display period DT is input according to an instruction of the medical provider received via the input interface 17 (step S207: Yes), the time series data in the display period is obtained by the obtaining function 193, so that the processes at step S201 and thereafter are repeatedly performed. The instruction to change the display period may be realized by a swipe operation performed on the display period DT illustrated in FIGS. 3 to 7, for example. Alternatively, instead of the swipe operation, the instruction to change the display period may be realized by manipulating a knob NB on a scroll bar SB related to changing the display period DT, as illustrated in FIGS. 5 and 6. Further, when arrows are displayed on the two ends of the scroll bar SB, the instruction to change the display period may be realized by pressing the arrows.

More specifically, when the display period DT has been changed, the identifying function 197 at step S203 identifies a state of the patient (hereinafter, “third state”) corresponding to the post-change display period. Subsequently, at step S204, the determining function 199 determines, on the basis of the time series data, reference data corresponding to a state of the patient (hereinafter, “fourth state”) that is different from the third state and is identified in accordance with the change made to the display period. After that, at step S205, the display controlling function 201 causes the display 15 to display the time series data corresponding to the post-change display period and the reference data corresponding to the fourth state, so as to be kept in correspondence with each other. As a result, the time series data displayed by the display 15 in the display period DT and the reference period have been updated.

When no change made to the type of diagnosis/treatment information displayed in the display period DT is input according to an instruction of the medical provider received via the input interface 17 (step S207: No), the process at step S208 will be performed. In this situation, the order in which the process at step S206 and the process at step S207 are performed may be reversed.

### Step S208:

When a prompt to finish the display of the time series data in the display period DT is input according to an instruction of the medical provider received via the input interface 17 (step S208: Yes), the state display process will end. On the contrary, when no prompt to finish the display of the time series data in the display period DT is input according to an instruction of the medical provider received via the input interface 17 (step S208: No), the process at step S206 will be performed.

The medical information display apparatus 1 according to the embodiment described above is configured: to obtain the time series data related to the predetermined patient; to receive the display period DT being a range of the data to be displayed from among the time series data; to identify the first state of the patient corresponding to the display period DT on the basis of the time series data; to determine the reference data corresponding to the second state different from the first state on the basis of the time series data; and to cause the display 15 to display the time series data corresponding to the display period DT and the reference data so as to be kept in correspondence with each other.

For example, the medical information display apparatus 1 according to the embodiment is configured to identify the first state by using at least one piece of diagnosis/treatment information in the time series data and to determine the reference data by using at least one piece of diagnosis/treatment information in the time series data. For the medical information display apparatus 1 according to the embodiment, for example, the diagnosis/treatment information includes one or both of a subjective symptom related to the patient and details of a diagnosis/treatment event related to the patient. For the medical information display apparatus 1 according to the embodiment, the diagnosis/treatment event may be, for example, an event of being admitted into and/or discharged from a hospital related to the patient.

Further, the medical information display apparatus 1 according to the embodiment may be configured to identify the first state, by inputting the plurality of pieces of diagnosis/treatment information to the trained model that receives an input of a plurality of pieces of diagnosis/treatment information in the display period DT and outputs one of the first and the second states; to determine the second state by inputting the plurality of pieces of diagnosis/treatment information in a time period different from the display period DT to the trained model; and to determine the time series data corresponding to the second state as the reference data.

Further, the medical information display apparatus 1 according to the embodiment is configured to cause the display 15 to further display the second state. For example, the medical information display apparatus 1 according to the embodiment is configured to cause the display 15 to display the data in the time period closest to the display period among the reference data.

Further, the medical information display apparatus 1 according to the embodiment may be configured to determine the reference data, while regarding a state farthest away from the first state in a diagnosis/treatment sense, as the second state. For example, the medical information display apparatus 1 according to the embodiment may be configured: to determine the plurality of second states; and to determine the reference data on the basis of the differences between the first state and the plurality of second states and the temporary closeness between the plurality of time periods respectively corresponding to the plurality of second states and the display period DT.

Further, when a change is made to the display period DT according to an instruction of the medical provider received via the input interface 17, the medical information display apparatus 1 according to the embodiment is configured: to identify the third state corresponding to the post-change display period; to determine, on the basis of the time series data, the reference data corresponding to the fourth state that is different from the third state and is identified in accordance with the change made to the display period; and to cause the display 15 to display the time series data corresponding to the post-change display period and the reference data corresponding to the fourth state, so as to be kept in correspondence with each other.

With the above configurations, by using the medical information display apparatus 1 according to the embodiment, it is possible to determine the reference data and the reference period on the basis of the time series data of the patient and to automatically cause the reference data and the time series data to be displayed in relation to the same type of diagnosis/treatment information. For example, even in the situation where an index such as an activity amount related to the patient for which a reference value has not clinically been established is obtained as the time series data from the wearable device 5 or the like, it is possible by using the medical information display apparatus 1 to determine the reference data and to display the reference data together with the time series data in such a manner that medical providers are able to easily interpret the patient's state. In other words, by using the medical information display apparatus 1 in the present example, it is possible to provide the medical providers with a system (the display mode) that makes it possible to easily compare the reference data with the time series data.

As explained above, by using the medical information display apparatus 1 according to the embodiment, medical providers are able to easily check the time series data (raw data) of the patient while referring to the reference data, in relation to the final assessment about the patient's signs of exacerbation. For example, by using the medical information display apparatus 1 in the present example, medical providers are able to assess the patient's state, even from diagnosis/treatment information of which knowledge related to relationships with patients' states is not available. Accordingly, the medical information display apparatus 1 in the present example is able to improve efficiency of medical providers in reviewing processes utilizing their knowledge, in relation to validity of the determination about the patient's signs of exacerbation.

### Modification Examples

In the present modification example, as the reference data, for example, time series data of another patient who has the same type of disease as the patient is used. In other words, the difference between a state display process in the present modification example and the state display process in the embodiment lies in that, from diagnosis/treatment information of the other patient, the data (hereinafter, “relevant data”) that is relevant to the other patient and is of the same type as the time series data displayed on the display 15 and indicating a state of the patient is used for determining the reference data. For example, the relevant data corresponds to time series data of diagnosis/treatment information of the same type as the diagnosis/treatment information indicated by the displayed time series data, with respect to the other patient having the same disease as the patient related to the time series data displayed on the display 15. More specifically, the identifying function 197 is configured to identify the relevant data from the diagnosis/treatment information of the other patient, on the basis of the purpose of the examination, the examined items, the examined site, additional information related to the examination, and/or the like related to the displayed time series data. For example, when only a “stable state” is displayed on the display 15 as the patient's state, the determining function 199 is configured to determine a time period corresponding to an “exacerbated state” of the other patient who has the “exacerbated state”, as a reference period. Further, the determining function 199 may determine reference data in a reference period corresponding to the second state, from time periods of a plurality of other patients. In that situation, the display controlling function 201 may cause the display 15 to display the reference data of the plurality of other patients in a mixed manner.

For example, at step S204 in FIG. 2, the processing circuitry 19 may determine, by employing the determining function 199, reference data corresponding to the second state different from the first state, on the basis of the relevant data that is of the same type as the time series data of the patient and is relevant to the other patient. The relevant data corresponds to the time series data (e.g., various types of examination results, clinical information such as a medical image or an image interpretation report, biological information, and/or the like that are kept in association with an electronic medical record of the other patient) of the other patient who has the same type of disease as the patient.

More specifically, the determining function 199 is configured to determine the reference data, by using at least one piece of diagnosis/treatment information in the relevant data. In that situation, for example, the diagnosis/treatment information includes one or both of: a subjective symptom related to the other patient; and details of a diagnosis/treatment event related to the other patient. In this situation, the diagnosis/treatment event is an event of being admitted into and/or discharged from the hospital related to the other patient.

Further, when the judgment result at step S206 in FIG. 2 is “Yes” or when the judgment result at step S207 in FIG. 2 is “Yes”, the processing circuitry 19 is configured, by employing the determining function 199, to determine reference data corresponding to the fourth state that is different from the third state and is identified in accordance with a change made to the display period, on the basis of the relevant data that is of the same type as the time series data of the patient and is relevant to the other patient, at step S204 illustrated in FIG. 2. In that situation, at step S205 in FIG. 2, the display controlling function 201 is configured to cause the display 15 to display the time series data corresponding to the post-change display period and the reference data corresponding to the fourth state so as to be kept in correspondence with each other.

Because the other processes in the state display process in the present modification example are the same as those in the state display process according to the embodiment, explanations thereof will be omitted. By using the medical information display apparatus 1 according to the present modification example, even when no reference data is present in the time series data related to the patient (e.g., for a predetermined time period since the start of the acquisition of the time series data), it is possible to determine the reference data by using the time series data of the other patient who has the same symptom or disease as the patient. Because the other advantageous effects of the present modification example are the same as those of the embodiment, explanations thereof will be omitted.

When technical concept of the embodiment of the present disclosure is realized as a medical information display processing method, the medical information display method includes: obtaining time series data related to a predetermined patient; receiving a display period DT being a range of data to be displayed from among the time series data; identifying a first state of the patient corresponding to the display period DT, on the basis of the time series data; determining reference data corresponding to a second state different from the first state, on the basis of one selected from between relevant data that is of the same type as the time series data and is relevant to another patient and the time series data; and causing the display 15 to display time series data corresponding to the display period DT and the reference data, so as to be kept in correspondence with each other. Because processing procedures and advantageous effects of the present medical information display method are the same as those of the embodiment, explanations thereof will be omitted.

When technical concept of the embodiment of the present disclosure is realized as a medical information display program, the medical information display program causes a computer to realize: obtaining time series data related to a predetermined patient; receiving a display period DT being a range of data to be displayed from among the time series data; identifying a first state of the patient corresponding to the display period DT, on the basis of the time series data; determining reference data corresponding to a second state different from the first state, on the basis of one selected from between relevant data that is of the same type as the time series data and is relevant to another patient and the time series data; and causing the display 15 to display time series data corresponding to the display period DT and the reference data, so as to be kept in correspondence with each other. In that situation, the program capable of causing the computer to implement the method may also be distributed as being stored in a storage medium such as a magnetic disk (e.g., a hard disk), an optical disc (e.g., a Compact Disc Read-Only Memory (CD-ROM) or a Digital Versatile Disc (DVD)), or a semiconductor memory. Because processing procedures and advantageous effects of the medical information display program are the same as those of the embodiment, explanations thereof will be omitted.

According to at least one aspect of the embodiments and the like described above, it is possible to display the reference information together with the time series information in such a manner that medical providers are able to easily interpret the patient's state.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information display apparatus comprising processing circuitry configured:
to obtain time series data related to a predetermined patient;
to receive a display time period being a range of data to be displayed from among the time series data;
to identify a first state of the patient corresponding to the display time period, on a basis of the time series data;
to determine reference data corresponding to a second state different from the first state, on a basis of one selected from between relevant data that is of a same type as the time series data and is relevant to another patient and the time series data; and
to cause a display to display time series data corresponding to the display time period and the reference data, so as to be kept in correspondence with each other.

2. The medical information display apparatus according to claim 1, wherein the processing circuitry is configured:
to identify the first state by using at least one piece of diagnosis/treatment information in the time series data; and
to determine the reference data by using at least one piece of diagnosis/treatment information in the one selected from between the time series data and the relevant data.

3. The medical information display apparatus according to claim 2, wherein the diagnosis/treatment information includes one or both of: a subjective symptom related to the patient or said another patient; and details of a diagnosis/treatment event related to the patient or said another patient.

4. The medical information display apparatus according to claim 3, wherein the diagnosis/treatment event is an event of being admitted into and/or discharged from a hospital related to the patient or said another patient.

5. The medical information display apparatus according to claim 1, wherein the processing circuitry is configured:
to identify the first state, by inputting a plurality of pieces of diagnosis/treatment information to a trained model that receives an input of a plurality of pieces of diagnosis/treatment information in the display time period and outputs one of the first and the second states;
to determine the second state by inputting, to the trained model, a plurality of pieces of diagnosis/treatment information in a time period different from the display time period; and
to determine time series data corresponding to the second state as the reference data.

6. The medical information display apparatus according to claim 1, wherein the processing circuitry is configured to cause the display to further display the second state.

7. The medical information display apparatus according to claim 1, wherein the processing circuitry is configured to cause the display to display data in a time period closest to the display time period among the reference data.

8. The medical information display apparatus according to claim 1, wherein the processing circuitry is configured to determine the reference data, while regarding a state farthest away from the first state in a diagnosis/treatment sense, as the second state.

9. The medical information display apparatus according to claim 1, wherein the processing circuitry is configured
to determine a plurality of second states; and
to determine the reference data, on a basis of differences between the first state and the plurality of second states and temporary closeness between a plurality of time periods respectively corresponding to the plurality of second states and the display time period.

10. The medical information display apparatus according to claim 1, wherein
the processing circuitry is configured:
to identify, when the display time period is changed, a third state corresponding to a post-change display time period;
to determine reference data corresponding to a fourth state that is different from the third state and is identified in accordance with the change made to the display time period, on a basis of one selected from between relevant data that is of a same type as the time series data and is relevant to another patient and the time series data; and
to cause the display to display time series data corresponding to the post-change display time period and the reference data corresponding to the fourth state so as to be kept in correspondence with each other.

11. A medical information display method comprising:
obtaining time series data related to a predetermined patient;
receiving a display time period being a range of data to be displayed from among the time series data;
identifying a first state of the patient corresponding to the display time period, on a basis of the time series data;
determining reference data corresponding to a second state different from the first state, on a basis of one selected from between relevant data that is of a same type as the time series data and is relevant to another patient and the time series data; and
causing a display to display time series data corresponding to the display time period and the reference data, so as to be kept in correspondence with each other.

12. A non-volatile computer-readable storage medium storing therein a medical information display program that causes a computer to realize:
obtaining time series data related to a predetermined patient;
receiving a display time period being a range of data to be displayed from among the time series data;
identifying a first state of the patient corresponding to the display time period, on a basis of the time series data;
determining reference data corresponding to a second state different from the first state, on a basis of one selected from between relevant data that is of a same type as the time series data and is relevant to another patient and the time series data; and
causing a display to display time series data corresponding to the display time period and the reference data, so as to be kept in correspondence with each other.
